(19) United States
(12) Patent Application Publication
(10) Pub. No.: US 2023/0215514 A1
(43) Pub. Date: Jul. 6, 2023

(54) METHOD FOR ANALYZING GENETIC INTERACTION OF CANCER VIA MOLECULAR NETWORK REFINING PROCESS, AND SYSTEM USING SAME

(71) Applicant: INDUSTRY FOUNDATION OF CHONNAM NATIONAL UNIVERSITY, Gwangju (KR)

(72) Inventors: Jin Myung JUNG, Chungcheongnam-do (KR); Sun Yong YOO, Gwangju (KR)

(73) Assignee: INDUSTRY FOUNDATION OF CHONNAM NATIONAL UNIVERSITY, Gwangju (KR)

(21) Appl. No.: 17/968,902

(22) Filed: Oct. 19, 2022

(30) Foreign Application Priority Data
Jan. 5, 2022 (KR) 10-2022-0001820

(51) Int. Cl. G16B 25/10; G16B 20/20; G16B 5/00

(52) U.S. Cl. CPC G16B 25/10; G16B 20/20; G16B 5/00

(57) ABSTRACT

Disclosed herein are a method for analyzing a genetic interaction to reduce a false positive in gene screening for at least one gene cluster associated with at least one type of cells by deriving the genetic interaction and a synthetic partner with at least one profile selected from the group consisting of a mutation profile, a loss-of-function profile, and an expression profile; and a system using same.

### CROSS-REFERENCE TO RELATED APPLICATION

This application is based on and claims priority under 35 U.S.C. 119 to Korean Patent Application No. 10-2022-0001820, filed on Jan. 5, 2022, in the Korean Intellectual Property Office, the disclosure of which is herein incorporated by reference in its entirety.

### FIELD

The present disclosure relates to: a method for analyzing a genetic interaction to reduce a false positive in gene screening for at least one gene cluster associated with at least one type of cells by deriving the genetic interaction and a synthetic partner with at least one profile selected from the group consisting of a mutation profile, a loss-of-function profile, and an expression profile; and a system using same.

### BACKGROUND

The identification of cancer-essential genes specific to a certain mutated gene, also referred to as synthetic lethal interactions (SLI), is crucial for establishing therapeutic strategies and understanding the mechanisms of cancer. Inhibiting genes that are synthetically lethal to a certain mutation would kill cancer cells harboring such mutations while sparing normal cells, which could facilitate/develop precision medicine. For example, the PARP1 gene has been proven to be an essential gene specific to mutated BRCA (i.e., synthetically lethal to mutated BRCA), and the use of the PARP1 inhibitor olaparib was approved for treating BRCA-mutated ovarian cancer. On the other hand, cancer suppressor genes specific to a certain mutated gene also provide opportunities for cancer therapeutics. Cancer cells harboring a certain mutation can be killed via the activation (or upregulation) of the suppressor genes specific to the mutation, even though this approach is more challenging than inhibiting essential genes.

GIs are typically characterized by loss-of-function perturbation using CRISPR and RNAi. Statistical analysis of cancer growth after knocking out/down genes by CRISPR/RNAi in multiple cancer cells yields quantitative assessments of GIs. To date, many research groups have systematically identified GIs at the genome scale by performing high-throughput loss-of-function screening on a panel of cancer cell lines using CRISPR or RNAi. However, CRISPR and RNAi techniques have their own limitations and yield considerable false positives in the identification of GIs. For example, knockout by CRISPR sometimes induces cell death mediated by the DNA damage response irrespective of target gene inhibition. In addition, the RNAi approach involves off-target effects that silence the mRNA molecules of unintended targets. These are probably the reasons that few GIs have been reproduced across multiple studies. In addition, large-scale multiple testing may be another factor contributing to the false positives. Multiple testing is necessary to analyze loss-of-function screening data for thousands of genes performed on cells containing thousands of mutations, but it inherently leads to considerable false positives.

There is therefore a need for a method for analysis of genes that can exceptionally reduce false positives when screening massive genes for multiple cells.

### SUMMARY

In the present disclosure, two kinds of processes were newly devised and applied to decrease false prediction in characterizing GIs by applying constraints that consider actual biological phenomena.

First, loss-of-function data of non-expressed genes were excluded in characterizing GIs, under the assumption that they would not affect cell systems. The present inventors noticed that one out of six of the analyzed genes in the disclosure was non-expressed, and knockout/knockdown of non-expressed genes theoretically should not influence any cell processes. This means that technical defects, such as off-target effects, exist if their depletion scores are not trivial.

Second, more importantly, the characterized GIs were refined by utilizing molecular networks such as Kyoto Encyclopedia of Genes and Genomes (KEGG) and protein-protein interaction (PPI) network analysis, under the assumption that genes genetically interacting with a certain mutated gene are located adjacently in the networks. The second assumption is derived from the fact that a chemical signal is transmitted through a cell as a series of biochemical events on molecular networks, which ultimately results in a cellular process, such as cell proliferation or apoptosis (i.e., signal transduction).

In the present disclosure, the two kinds of processes introduced above are newly employed to decrease the occurrence of false predictions, whereby the refining process (RP) based on molecular networks yields a synthetic partner network (SPN) for each mutated gene, which will provide good insight into the mechanism or therapeutics of cancer. The results were evaluated for the previously known synthetic lethal interactions in the two datasets from MiSL and synlethDB, which allows improved precision in most comparisons. Therefore, the present disclosure is expected to reduce false GI characterizations and provide assistance in cancer research.

Accordingly, an aspect of the present disclosure is to provide a method for analyzing a genetic interaction.

Another aspect of the present disclosure is to provide a genetic interaction analysis system including at least one processor that operates to execute computer-readable instructions.

A further aspect of the present disclosure is to provide a system for executing a computer program recoded on a computer-readable medium to implement the method for analyzing a genetic interaction.

The present disclosure relates to a method for analysis of a genetic interaction and a system using same and, more specifically, to a method for analysis of a genetic interaction and a system using same, wherein a genetic interaction and a synthetic partner are derived using at least one profile selected from the group consisting of a mutation profile, a loss-of-function profile, and an expression profile, thereby decreasing a false positive in gene screening for at least one gene cluster associated with at least one type of cells.

Below, a detailed description will be given of the present disclosure.

An aspect of the present disclosure is drawn to a method for analysis of a genetic interaction, the method including: a first profile input step; a second profile input step; a data mapping step; and a refining step.

In the present disclosure, the first profile input step may be adapted to input a gene dataset including at least one profile selected from the group consisting of a mutation profile, a loss-of-function profile, and an expression profile, for at least one gene cluster associated with at least one type of cells, but with no limitations thereto.

In an embodiment of the present disclosure, the first profile input step may be adapted to input a gene dataset, including a mutation profile, a loss-of-function profile, and an expression profile, for at least one gene cluster associated with at least one type of cells.

In an embodiment of the present disclosure, the gene dataset of the first profile input step may be input from a DepMap database, with no limitations thereto.

In an embodiment of the present disclosure, the mutation profile may include about 1,300,000 mutation events for 18,000 genes across 1,741 cell lines, but with no limitations thereto.

In an embodiment of the present disclosure, the mutation profile may include at least one variant type selected from the group consisting of de novo start out of frame, frame shift deletion, frame shift insertion, in-frame deletion, nonsense mutation, nonstop mutation, splice site, start codon deletion, start codon insertion, stop codon deletion, stop codon insertion, and missense mutation and may include all of the 12 variant types, with no limitations thereto.

In the present disclosure, the loss-of-function profile may include a depletion score.

In the present disclosure, the depletion score may mean a number of cells that are alive when a certain gene is knocked out/down by loss-of-function screening.

In this regard, a low depletion score of a certain gene (i.e., the gene is depleted or underrepresented) may mean that most cells in which the gene is knocked out/down by CRISPR/RNAi are dead, indicating that the knockout/down of the gene induces cancer death.

In the present disclosure, the expression profile may include gene expression events for 19,000 genes across 1,305 cell lines.

In the present disclosure, the second profile input step may be adapted to input a network set for mapping a gene dataset to construct a network.

In the present disclosure, the network set may include at least one selected from the group consisting of a genetic interaction network profile and a protein-protein interaction network profile, for example, both of the network profiles, but with no limitations thereto.

In the present disclosure, the genetic interaction network profile may be input from at least one database selected from the group consisting of KEGG pathway, SIGnature DataBase, Gene ontology, Consortium, DisGeNET, and Diseases, for example, from KEGG pathway, but with no limitations thereto.

In an embodiment of the present disclosure, the genetic interaction network profile may include, but is not limited to, signal transduction pathways, cancer pathways, and cell growth-related pathways. In an embodiment of the present disclosure, the genetic interaction network profile may include 47 KEGG pathways, but is not limited thereto.

In an embodiment of the present disclosure, the genetic interaction network profile may consist of directed edge.

In the present disclosure, the protein-protein interaction (PPI) network profile may be input from BIOGRID database, but with no limitations thereto.

In an embodiment of the present disclosure, the protein-protein interaction (PPI) network profile may consist of undirected edge.

In the present disclosure, the protein-protein interaction network profile may include a protein interaction discovered by affinity chromatography technology or a two-hybrid detection method, but with no limitations thereto.

In the present disclosure, the data mapping step may be adapted to map a gene dataset to a network set to generate a genetic interaction data.

In the present disclosure, the mapping may be a process of identifying: a sensitive genetic interaction (GI) between a mutated first gene and a second gene in a test cell group when knockout/knockdown of the second gene induces death of cancer cells compared to control cells; and a resistant genetic interaction (GI) between a mutated first gene and a second gene in a test cell group when knockout/knockdown of the second gene induces proliferation of cancer cells or blocks death of cancer cells, but does not show such results for control cells.

In the present disclosure, the mapping may include a process of excluding a depletion score for a non-expressed gene by using the expression profile, but with no limitations thereto.

In the present disclosure, the mapping may include a process of excluding a non-expressed gene from the mapping by using the expression profile, but with no limitations thereto.

In the present disclosure, the depletion score for a gene may mean a number of cells that are alive when the gene is knocked out or down.

In the present disclosure, the refining process may be adapted to exclude a synthetic partner for a specific mutant gene from the network profile in the genetic interaction data when the synthetic partner for the specific mutant gene is not located within a predetermined genetic distance from a different synthetic partner for the specific mutant gene on the genetic interaction data.

In the present disclosure, the predetermined genetic distance may mean a distance from any synthetic partner for a specific mutant gene to a different synthetic partner interacting therewith on the genetic interaction data.

In the present disclosure, the predetermined genetic distance may be at least one selected from the group consisting of 1, 2, 3, 4, and 5, for example, may be 1 and 2, but is not limited thereto.

When the genetic distance exceeds 5, there are many different interacting synthetic partners that exhibit complex interactions, making it difficult to derive a potential therapeutic target gene.

In the present disclosure, the method for analyzing a genetic interaction may further include a target gene deriving step for deriving a potential therapeutic target gene after the refining step.

In the present disclosure, the target gene deriving step may be adapted to derive, as a potential therapeutic target gene, a gene interacting with a certain gene on the genetic interaction data to which the refining step has been applied.

Contemplated according to an aspect of the present disclosure is a system for analyzing a genetic interaction, the system including at least one processor that operates to execute computer-readable instructions, wherein the at least one processor is adapted to:

receive a gene dataset, including at least one profile selected from the group consisting of a mutation profile a loss-of-function profile, and an expression profile, for at least one gene cluster associated with at least one type of cells;

receive a network set;

map the gene dataset to the network set to generate genetic interaction data; and

exclude a synthetic partner for a specific mutant gene from the network profile in the genetic interaction data when the synthetic partner for the specific mutant gene is not located within a predetermined genetic distance from a different synthetic partner for the specific mutant gene on the genetic interaction data.

A further aspect of the present disclosure is drawn to a computer program recorded on a computer-readable medium to implement a method for analyzing a genetic interaction, the method including:

a first profile input step of inputting a gene dataset including at least one profile selected from the group consisting of a mutation profile, a loss-of-function profile, and an expression profile, for at least one gene cluster associated with at least one type of cells;

a second profile input step of inputting a network set;

a data mapping step of mapping the gene dataset to a network set to generate genetic interaction data; and

a refining step of excluding a synthetic partner for a specific mutant gene from the network profile in the genetic interaction data when the synthetic partner for the specific mutant gene is not located within a predetermined genetic distance from a different synthetic partner for the specific mutant gene on the genetic interaction data.

In an embodiment of the present disclosure, the computer program may independently or collectively instruct or configure the processing device to operate as desired. The computer program may be embodied permanently or temporarily in any type of machine, component, physical or virtual equipment, computer storage medium or device capable of providing instructions or data to or being interpreted by the processing device. The software also may be distributed over network coupled computer systems so that the software is stored and executed in a distributed fashion. The computer program may be stored by one or more non-transitory computer readable recording mediums.

The methods according to the above-described embodiments may be recorded in non-transitory computer-readable media including program instructions to implement various operations embodied by a computer. The media may continuously store a program executable by a computer or may temporarily store or the program for execution or download. Also, the media may be various types of recording devices or storage devices in which a single piece or a plurality of pieces of hardware may be distributed over a network without being limited to a medium directly connected to a computer system. Examples of the media may include magnetic media such as hard disks, floppy disks, and magnetic tapes; optical media such as CD-ROM discs and DVDs; magneto-optical media such as floptical disks; and hardware devices that are specially configured to store and perform program instructions, such as read-only memory (ROM), random access memory (RAM), flash memory, and the like. Examples of other media may include recording media and storage media managed at Appstore that distributes applications, sites and servers that supply and distribute various types of software, and the like.

The program instructions recorded on the media may be those specially designed and constructed for the purposes of the embodiments, or they may be of the kind well-known and available to those having skill in the computer software arts. Examples of program instructions include both machine code, such as produced by a compiler, and files containing higher level code that may be executed by the computer using an interpreter.

The present disclosure relates to: a method for analyzing a genetic interaction to reduce a false positive in gene screening for at least one gene cluster associated with at least one type of cells by deriving the genetic interaction and a synthetic partner with at least one profile selected from the group consisting of a mutation profile, a loss-of-function profile, and an expression profile; and a system using same, whereby the present disclosure can provide new aspects of GI characterization.

With the ability to provide a handful of potentially rationale therapeutic targets for in-vitro and in-vivo experiments that consume a lot of time and costs, the present disclosure can decrease false positives, provide assistance to in vivo and in vitro experiments, and further improve economic benefit and efficiency in the research into therapeutics for diseases such as cancer.

### BRIEF DESCRIPTION OF THE DRAWINGS

The above and other aspects, features and advantages of the present disclosure will be more apparent from the following detailed description taken in conjunction with the accompanying drawings, in which:

FIG. 1 is a schematic view illustrating overall strategy for analysis of genetic interactions according to the present disclosure:

FIG. 2 shows distributions of the number of mutated genes in mutation profiles in terms of violin plots and histograms;

FIG. 3 shows distributions of the number of depleted genes of cell lines in loss-of function profiles obtained from CRISPR knockout screening in terms of violin plots and histograms;

FIG. 4 shows distributions of the number of depleted genes of cell lines in loss-of function profiles obtained from shRNA knockout screening in terms of violin plots and histograms;

FIG. 5 shows distributions of the number of non-expressed genes of cell lines in expression profiles in terms of violin plots and histograms.

FIG. 6 shows violin plots accounting for whether depletion scores of the knocked-out gene for normal and mutated cells are consistent according to the use of the exclusion process;

FIG. 7 shows the number of genetic interactions for each mutated gene refined based on KEGG networks from CRISPR screening and shRNA screening;

FIG. 8 shows synthetic partner network 1 (SPN1) and synthetic partner network 2 (SPN2) for mutated BRAF in KEGG networks from CRISPR screening;

FIG. 9 shows bar graphs of the number of refined genetic interactions based on PPI networks from CRISPR screening and shRNA screening in which only the mutated genes with five or more initial GIs are presented among 789 and 570 initial GIS for CRISPR screening and shRNA screening, respectively;

FIG. 10 shows SPN1 for mutated BRAF based on PPI networks from CRISPR screening;

FIG. 11 shows bar graphs in which 480 sensitive GIs with exclusion procedure (SGWE) and 519 sensitive GIs without exclusion procedure (SGOE) from shRNA screening are compared;

FIG. 12 shows bar graphs in which the sensitive GIs without application of any of RP (INIT), RP2, and RP1 are evaluated for recall and precision;

FIG. 13 shows bar graphs of the precision of sensitive GIs without any of RP, RP2, and RP1 in which sensitive GIS refined based on KEGG/PPI networks are evaluated with the synlethDB and MISL; and

FIG. 14 shows PPI networks for mutated NRAS from CRISPR screening.

### DETAILED DESCRIPTION

The present disclosure may be variously modified and include various exemplary embodiments in which specific exemplary embodiments will be described in detail hereinbelow. However, it shall be understood that the specific exemplary embodiments are not intended to limit the present disclosure thereto and cover all the modifications, equivalents and substitutions which belong to the idea and technical scope of the present disclosure.

Genetic interactions (GIs), such as synthetic lethal interaction (SLI), are promising therapeutic targets in precision medicine. However, despite extensive efforts to characterize GIs by large-scale perturbation screening, considerable false positives have been reported in multiple studies.

The present disclosure proposes a new computational approach for improved precision in identifying GIs by applying constraints that consider actual biological phenomena. In the present disclosure, GIs are characterized by assessing mutation, loss of function, and expression profiles in the DEPMAP database. The expression profiles are used to exclude loss-of-function data for non-expressed genes in GI characterization. More importantly, the characterized GIs are refined based on Kyoto Encyclopedia of Genes and Genomes (KEGG) or protein-protein interaction (PPI) networks, under the assumption that genes genetically interacting with a certain mutated gene are adjacent in the networks.

As a result, initial GIs characterized with CRISPR and RNAi screenings were refined to 65 and 23 GIs based on KEGG networks and to 183 and 142 GIs based on PPI networks, respectively. The evaluation of refined GIs shows improved precision with respect to known synthetic lethal interactions. The refining process also yields a synthetic partner network (SPN) for each mutated gene, which provides insight into therapeutic strategies for the mutated genes; specifically, exploring the SPN of mutated BRAF revealed ELAVL1 as a potential target for treating BRAF-mutated cancer, as validated by previous research. According to the present disclosure, this work is expected to advance cancer therapeutic research.

### 1. OVERVIEW OF THE PRESENT DISCLOSURE

Mutation profiles of 18,000 genes across 1747 cell lines were obtained from DepMap, and they were marked as functional mutations if deleterious, such as frame shifts, stop codon deletions, and missense mutations. Out of 18,000 genes, 4000 recurrently mutated genes, i.e., functionally mutated in more than 3% of the considered cell lines, were analyzed in the present disclosure.

Next, the depletion scores by loss of functions from CRISPR knockout screening (16,000 genes across 769 cell lines) and shRNA knockdown screening (6000 genes across 702 cell lines) were individually acquired from DepMap. The GI between a mutated gene Q and a gene K was characterized when the knockout/knockdown of gene K statistically caused cancer death or proliferation in cells harboring mutated gene Q, which was executed by applying t-tests to the depletion score of gene K between cells with mutated gene Q and normal gene Q (FDR<0.2).

In the present disclosure, the excluding and refining processes were newly recruited to diminish false GIs.

First, the depletion scores of non-expressed genes were excluded in the t-tests, with the assumption that knockout/knockdown of non-expressed genes would not affect cell systems. Second, the characterized GIs were further refined by incorporating the KEGG or PPI networks. The assumption was that synthetic partners (SPs) of a certain mutated gene are located adjacent in the networks.

The refining process also provides a synthetic partner network (SPN) for a certain mutated gene, which can be used to research the mechanism or therapeutic strategy of the mutated gene. The characterized GIs were evaluated for previously known synthetic lethal interactions in the two datasets from MiSL and synlethDB.

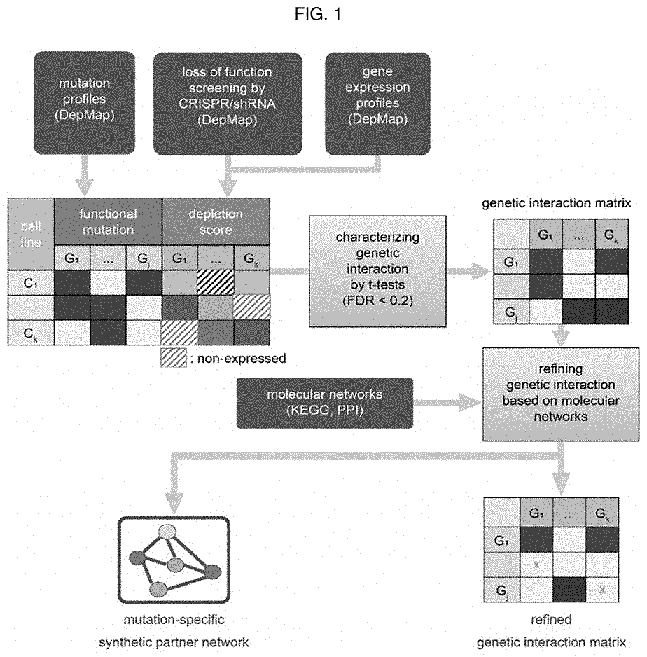

The strategy overview of the present disclosure is illustrated in FIG. 1.

In the present disclosure, a cancer-essential (suppressor) gene K specific to a mutated gene Q is referred to as a sensitive (resistant) genetic interaction (GI) between the mutated gene Q and the gene K.

With reference to FIG. 1, the genetic interaction (GI) between a mutated gene Q and a gene K was characterized when the knockout/knockdown of gene K statistically caused cancer death or proliferation in cells harboring mutated gene Q. To quantitatively estimate this characterization, a t-test was applied to depletion scores from loss-of-function screening (CRISPR and shRNA) between cells with mutated and normal genes, where the depletion scores of non-expressed genes were excluded.

In addition, the characterized GIs were further refined by incorporating the KEGG or PPI networks based on the assumption that genes genetically interacting with a certain mutated gene (i.e., synthetic partners of a certain mutated gene) are located adjacent in the networks. The objective of these two processes was to diminish potential false predictions.

As a result, a refined set of GIs and a synthetic partner network (SPN) for each mutated gene were obtained.

### 2. METHODS

2-1. Data Preprocessing

2-1-1. Mutation Profiles

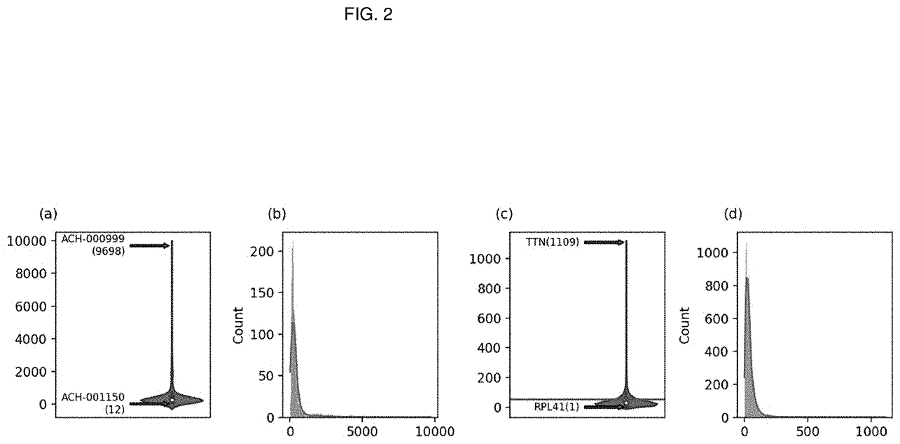

Mutation profiles named ‘CCLE_mutation.csv’ in the DepMap (Broad Institute) database contain ca. 1,300,000 mutation events for 18,000 genes across 1741 cell lines. A variant type was assigned to each mutation event, and among 20 variant types, 12 types, including de novo start out of frame, frame shift deletion, frame shift insertion, in-frame deletion, nonsense mutation, nonstop mutation, splice site, start codon deletion, start codon insertion, stop codon deletion, stop codon insertion, and missense mutation, were considered deleterious. A certain gene is determined to be functionally mutated if associated with even one deleterious mutation. The distributions of the number of mutated genes across cell lines are depicted in FIG. 2. Out of 18,000 genes, only 4000 genes functionally mutated in more than 3% of the cell lines (i.e., recurrently mutated genes) were analyzed in the present disclosure.

2-1-2. Loss-of-Function Profiles

In loss-of-function screening, the depletion score of a gene indicates the number of surviving cells when the gene is knocked out/down. Simply, a low depletion score of a certain gene (i.e., the gene is depleted or underrepresented) means that most cells in which the gene is knocked out/down by CRISPR/RNAi are dead, indicating that the knockout/down of the gene induces cancer death.

On the other hand, the high depletion score of a gene (i.e., the gene enriched or overrepresented) implies that most cells in which the gene is knocked out/down by CRISPR/RNAi are alive, supporting that the knockout/knockdown of the gene contributes to cancer proliferation or blocks cancer death.

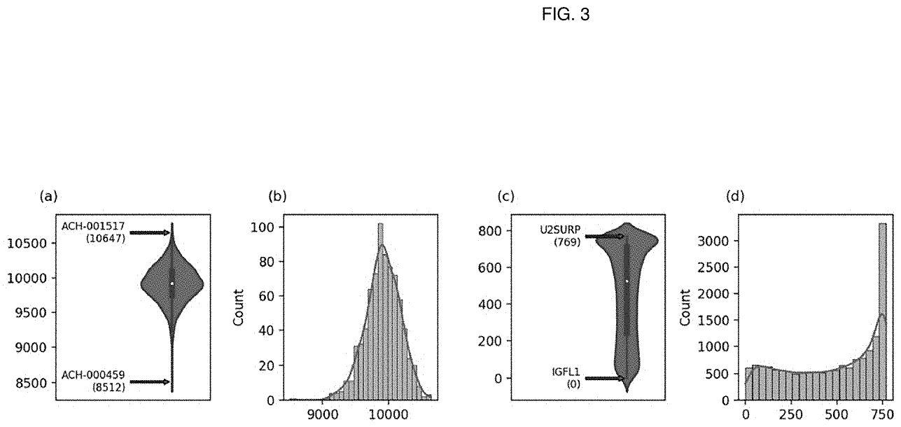

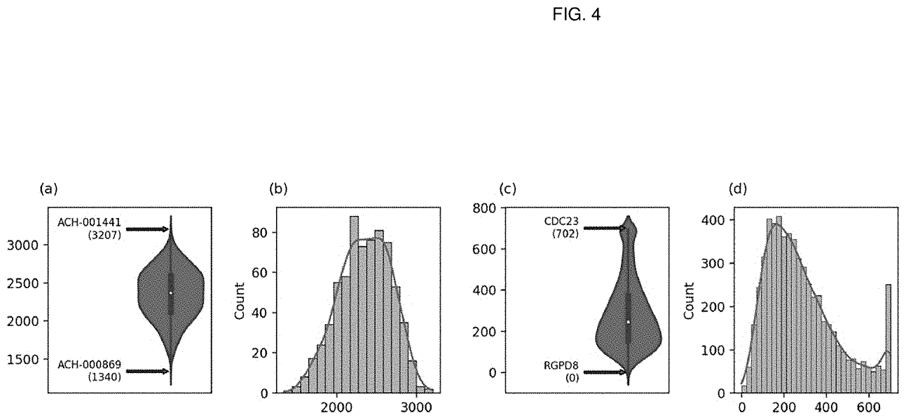

These loss-of-function pooled screenings are typically performed with CRISPR or shRNA genome-wide libraries. From the DepMap database, the two profiles of depletion scores named ‘Achillesgene_effect.csv’ (performed in CRISPR knockout screening) and ‘D2_combinedgene_dep_scores.csv’ (performed in shRNA knockdown screening) were acquired. The distributions of the number of depleted genes across cell lines are depicted in FIGS. 3 and 4. After removing all missing values, depletion scores were obtained for 16,000 genes across 769 cell lines from CRISPR screening and for 6000 genes across 702 cell lines from shRNA screening.

2-1-3. Expression Profiles

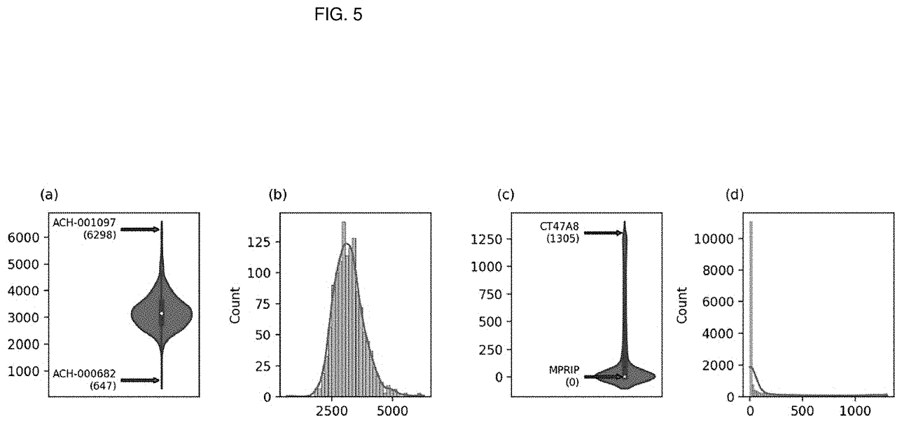

Expression profiles were also obtained from the DepMap database for 19,000 genes across 1,305 cell lines. Noticeably, it was observed that 4,000,000 records were zero (i.e., non-expressed) among all 25,000,000 gene expression records. From a gene perspective, 24 genes (such as CT47A8, F8A2, and USP17L25) were non-expressed in all 1305 considered cell lines, and 1637 genes were non-expressed in more than 1000 of the 1305 cell lines. The distributions of the number of non-expressed genes across cell lines are depicted in FIG. 5.

2-1-4. Network Construction

Two kinds of molecular networks, i.e., KEGG and PPI networks, were constructed to refine the characterized GIs.

First, KEGG networks were constructed by integrating 47 KEGG pathways, such as signal transduction pathways, cancer pathways, and cell growth-related pathways, and are summarized in Table 1, below.

TABLE 1

| No. | Pathway |
|---|---|
| 1 | hsa04010(MAPK) |
| 2 | hsa04012(ErbB) |
| 3 | hsa04014(Ras) |
| 4 | hsa04015(Rap1) |
| 5 | hsa04020(Calcium) |
| 6 | hsa04022(cGMP_PKG) |
| 7 | hsa04024(cAMP) |
| 8 | hsa04064(NFKB) |
| 9 | hsa04066(HIF1) |
| 10 | hsa04068(FOXO) |
| 11 | hsa04071(Sphingol) |
| 12 | hsa04072(Phospho_D) |
| 13 | hsa04110(Cell_cycle) |
| 14 | hsa04115(p53) |
| 15 | hsa04150(mTOR) |
| 16 | hsa04151(PI3K_AKT) |
| 17 | hsa04152(AMPK) |
| 18 | hsa04210(Apoptosis) |
| 19 | hsa04216(Ferroptosis) |
| 20 | hsa04217(Necroptosis) |
| 21 | hsa04310(Wnt) |
| 22 | hsa04330(Notch) |
| 23 | hsa04340(Hedgehog) |
| 24 | hsa04350(TGF_beta) |
| 25 | hsa04370(VEGF) |
| 26 | hsa04371(Apelin) |
| 27 | hsa04390(Hippo) |
| 28 | hsa04630(JAK_STAT) |
| 29 | hsa04668(TNF) |
| 30 | hsa05200(pathways_in_cancer) |
| 31 | hsa05210(colorectal) |
| 32 | hsa05211(RCC) |
| 33 | hsa05212(pancreatic) |
| 34 | hsa05213(Endometrial) |
| 35 | hsa05214(Glioma) |
| 36 | hsa05215(Prostate) |
| 37 | hsa05216(Thyroid) |
| 38 | hsa05217(BCC) |
| 39 | hsa05218(Melanoma) |
| 40 | hsa05219(Bladder) |
| 41 | hsa05220(CML) |
| 42 | hsa05221(AML) |
| 43 | hsa05222(SCLC) |
| 44 | hsa05223(NSCLC) |
| 45 | hsa05224(Breast) |
| 46 | hsa05225(hepato) |
| 47 | hsa05226(Gastric) |

In addition to Table 1, the integrated networks contained 12,617 interactions among 1,678 genes.

Second, from the BIOGRID database, PPI networks were constructed by integrating protein interactions discovered by the ‘affinity chromatography technology’ or ‘two hybrid’ detection method. The PPI networks provided 373,394 interactions among 18,179 genes.

Here, the KEGG networks consisted of directed edges, and the PPI networks consisted of undirected edges.

2-2. Characterizing GIs

The present inventors considered two kinds of GIs, i.e., sensitive and resistant GIs. First, a sensitive GI between a mutated gene Q and a gene K is characterized when the knockout/knockdown of gene K causes cancer death in the case cells (i.e., cells harboring a mutated gene Q) compared to the control cells (i.e., cells with normal gene Q). In this case, the depletion scores of gene Kin the case cells are lower than those in the control cells.

Second, a resistant GI between a mutated gene Q and a gene K is characterized when the knockout/knockdown of gene K causes cancer proliferation or blocks cancer death in the case cells, but not in the control cells. In this case, the depletion scores of gene Kin the case cells are higher than those in the control cells.

T-tests were used to statistically characterize sensitive and resistant GIs. In more detail, every possible pair between recurrently mutated genes (introduced in section 1-1-1) and loss-of-function screened genes (introduced in section 1-1-2) was assessed by applying a t-test to the depletion scores of a screened gene between case and control cells, and significant GIs were characterized (FDR<0.2).

Here, the present inventors noticed that there were numerous non-expressed genes in the assessed cells (as introduced in section 1-1-3) and the depletion scores of the non-expressed genes were excluded in the t-tests. In greater detail, depletion scores of a certain gene in the considered cell lines were processed based on the matched expression scores. The expression profiles were also obtained from DepMap database (see section 1-1-3), and DepMap cell IDs (i.e., a primary key) were used for the match process. Here, if the matched expression score is zero (i.e., non-expressed), its depletion score is ignored in the t-test for characterizing genetic interaction. If not, the depletion score is used as it is in the t-test (see the depletion score matrix in FIG. 1).

The excluding procedure was applied to diminish potential false predictions, with the assumption that the knockout/knockdown of non-expressed genes would not affect cell systems.

2-3. Refining GI Based on Molecular Networks

To decrease potential false positives, the refining process (RP) was further applied to the characterized GIs based on the assumptions that the SPs of a certain mutated gene are located adjacently on the molecular networks. Based on the KEGG or PPI networks, the RP was applied to every mutated gene whose number of SPs was two or more. In the process, the SP of a certain mutated gene remained only if there were any other SPs of the mutated gene within a certain distance on the networks, and two kinds of distance, i.e., a distance of 1 and a distance of 2, were applied.

As a result of the RP, a set of refined synthetic partner (SP) and synthetic partner network (SPN) was generated for a certain mutated gene. The SPN is the subnetwork containing only the remaining SPs of the mutated gene. SPNk indicates an SPN acquired by applying the RP with a distance of k. SPNs provide connectivity for the SPs of a certain mutated gene and their associated neighbors, which is advantageous in terms of inferring therapeutic potential of the mutated gene.

### 3. RESULTS

3-1. Characterized GI

Among 75,000,000 assessments in the CRISPR screenings, 1,740 GIs were identified (FDR<0.2) with the exclusion process for non-expressed genes. However, 1,623 GIs were characterized (FDR<0.2) without the exclusion procedure. The exclusion procedure removed the 167 potential false positives and augmented the 284 potential true positives.

In the same manner, among 35,000,000 assessments for the shRNA screenings, 1389 and 1459 GIs were determined (FDR<0.2) with and without the exclusion procedure, respectively.

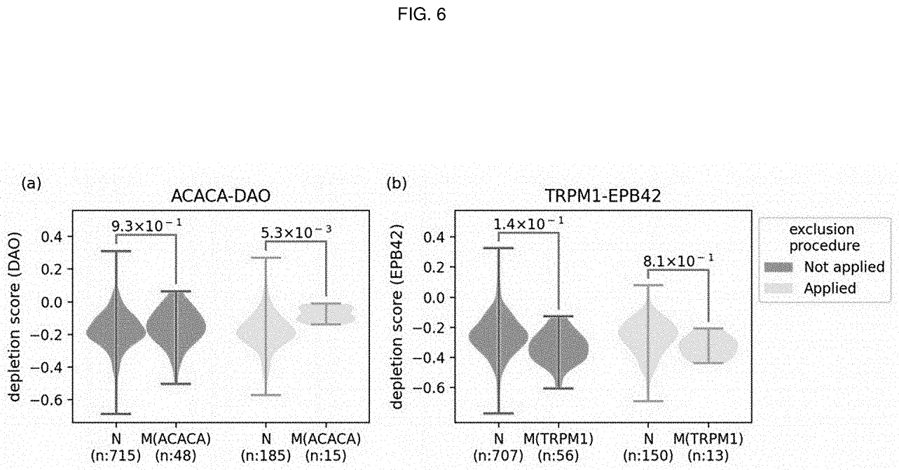

For a better understanding of the effect of the exclusion procedure, the depletion scores were illustrated in FIG. 6 as violin plots for the GIs whose significance was largely inconsistent according to the use of the exclusion procedure. In the figure, the numbers assigned to a pair of violin plots indicate FDR values of t-tests: N stands for normal cells (cells not harboring the mutation), M for mutated cells (cells harboring the mutation), and n for number of depletion scores.

As depicted in FIG. 6(a), a resistant GI between mutated ACACA and DAO was identified only when the exclusion procedure was applied.

On the other hand, as shown in FIG. 6(b), a sensitive GI between mutated TRPM1 and EPB42 was identified only when the exclusion procedure was not applied, indicating that it could be a potential false positive.

Notably, a decreased number of depletion scores was observed for the case where the exclusion procedure was applied. This is because the procedure ignores depletion scores of non-expressed genes when performing t-tests. As a result, 1,740 (418 sensitive and 1,322 resistant) and 1389 (480 sensitive and 909 resistant) GIs were characterized from CRISPR and shRNA screenings with the exclusion procedure, and these GIs are referred to as ‘original GIs’ in the present disclosure.

3-2. Refined GIs Based on KEGG Network Analysis

The original GIs were further refined based on the directed KEGG networks. To this end, all SPs of the original GIs were mapped to the 1,678 nodes on the KEGG networks, which yielded 525 and 417 mapped GIs in the CRISPR and shRNA screenings, respectively.

To apply the refining process (RP), the GIs of the mutated genes whose number of SPs was two or more were further narrowed down to 162 and 105 GIs, and these GIs were referred to as ‘initial GIs’. For example, in CRISPR screening, the 12 initial GIs of the mutated BRAF remained out of its 26 original GIs after mapping on the KEGG networks.

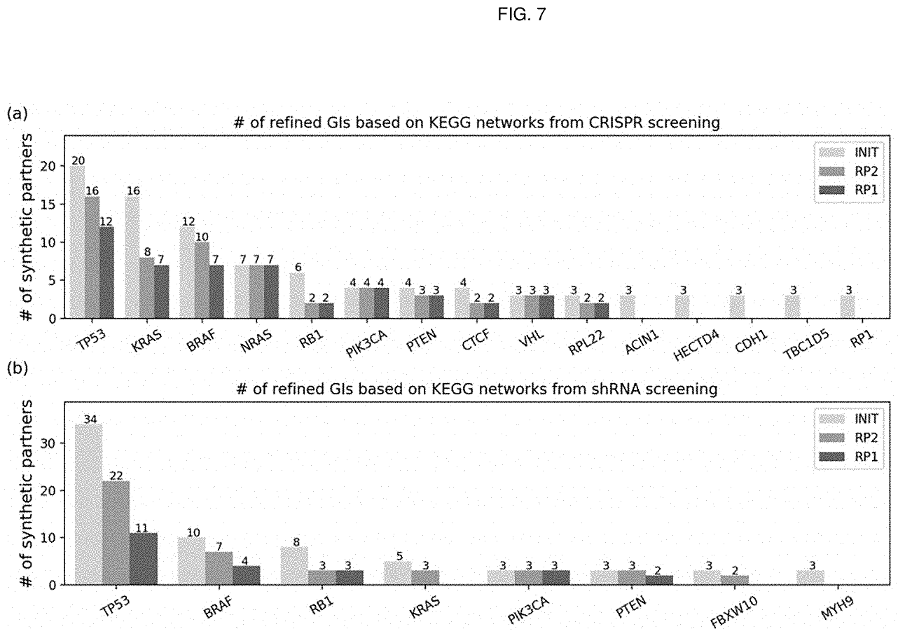

Then, the RP was applied to the initial GIs, and the numbers of GIs of each mutated gene, refined based on the KEGG networks from (a) CRISPR screening and (b) shRNA screening, are depicted in FIG. 7 (for clarity, only the mutated genes that had three or more initial GIs are presented; the x-axis indicates each mutated gene. INIT: the initial GIs after mapping on the KEGG networks (no RP was applied). RP2: GIs after applying the RP with a distance of 2 to the initial GIs. RP1: GIs after applying the RP with a distance of 1 to the initial GIs.)

As can be seen in FIG. 7, the 162 initial GIs in the CRISPR screenings were narrowed down to 73 and 65 GIs by applying the RP with distance 2 (RP2) and the RP with distance 1 (RP1), respectively. In the same manner, the 105 initial GIs in the shRNA screenings were narrowed down to 45 and 23 GIs by applying RP2 and RP1, respectively.

For example, in the CRISPR screening, the application of RP2 to mutated BRAF allowed 10 SPs out of the 12 initial SPs, which resulted from filtering out two initial SPs (SOX9 and ELAVL1). The application of RP1 yielded seven SPs by further filtering out three SPs (MDM2, PPP2R2A, and SHOC2).

For the KRAS mutation, out of the 16 initial SPs, eight (PTPN11, GRB2, SOS1, NRAS, RAF1, RRAS2, KRAS, and ITPR2) remained with RP2, and ITPR2 was additionally filtered out by RP1. On the other hand, in the case of NRAS mutation, no SPs were filtered out from the seven initial SPs (PTPN11, GAB1, SHOC2, GRB2, NRAS, RAF1, and KRAS) by RP2 and RP1, which means that all initial SPs were adjacent in the KEGG networks. For RB1 mutation, there were no changes in the remaining SPs between RP1 and RP2, i.e., SKP2 and CKS1B. For the five mutated genes (RP1, ACIN1, HECTD4, CDH1, and TBC1D5) in the CRISPR screening and one mutated gene (MYH9) in the shRNA screening, there were no remaining SPs after the RPs, which means that all the initial SPs were distant from each other.

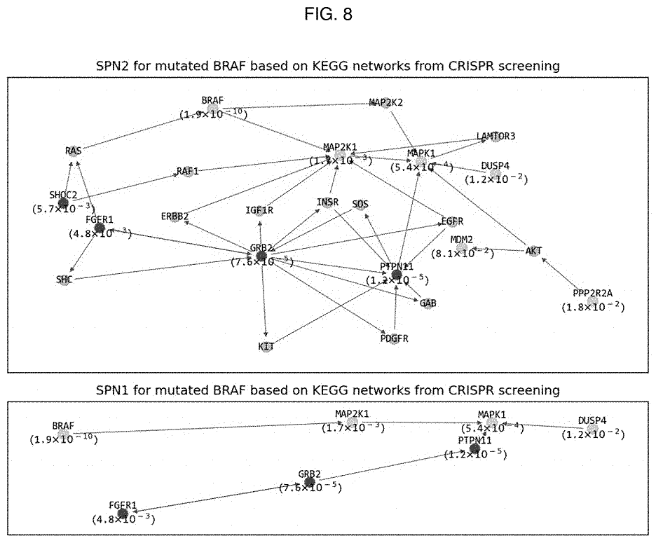

An SPN was also constructed for each mutated gene. For example, SPN2 and SPN1 for mutated BRAF from CRISPR screening are represented in FIG. 8 (the red and blue nodes are the SPs associated with GIs sensitive and resistant to mutated BRAF, respectively; the gray nodes are not SPs but genes connecting SPs of mutated BRAF; SPN2 includes the SPs that are connected to each other within a distance of 2, and SPN1 includes the SPs that adjoin each other; the number in parentheses for each SP indicates its FDR).

As seen in FIG. 8, SPN2 includes 10 SPs (red or blue nodes) and 14 neighbors connecting the SPs (gray nodes). Among the 10 SPs, six are sensitive SPs, i.e., BRAF, MAP2K1, MAPK1, DUSP4, MDM2, and PPP2R2A (represented as red nodes), and four are resistant SPs, i.e., SHOC2, FGFR1, GRB2, and PTPN11 (represented as blue nodes). Its own inhibition allowed the highest sensitivity (lowest FDR), which is consistent with the fact that BRAF is an oncogene addiction.

In SPN1, seven SPs were directly connected to each other, and the other three SPs (SHOC, PPP2R2A, and MDM2) were indirectly connected via neighbors (such as RAS and AK7). Interestingly, MAPK1 and PTPN11, which are contiguous SPs, present opposite types of GIs (sensitive and resistant) on the mutated BRAF. This can be explained by the fact that PTPN negatively regulates MAPK1.

Exploring SPNs can provide new therapeutic strategies. For example, by observing the SPNs of mutated BRAF, the present inventors noticed that inhibiting the six sensitive SPs or activating the four resistant SPs could be therapeutic strategies for precisely treating BRAF-mutated cancer. Furthermore, the 14 neighbors connecting the SPs (such as AKT, SOS, and RAF1) are other candidate therapeutic targets, which might be indicated as false negative results.

3-3. Refined GIs Based on PPI

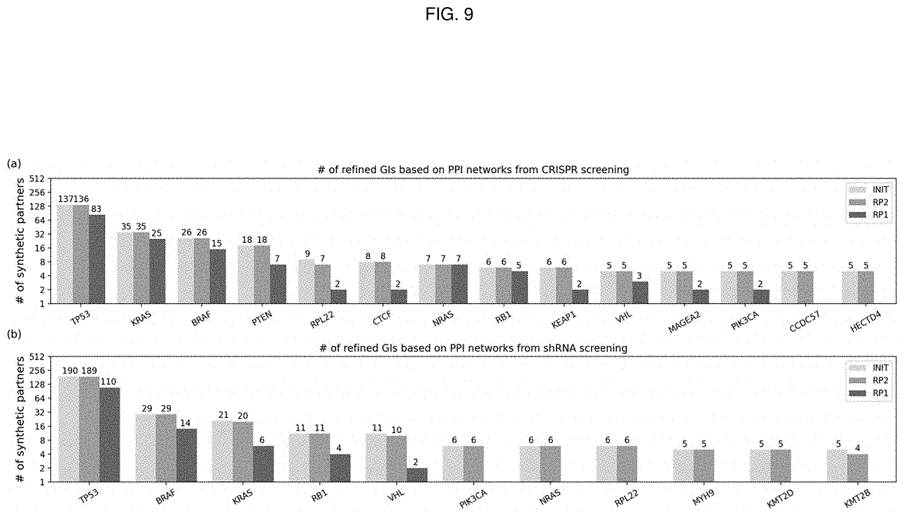

The original GIs were also refined based on undirected PPI networks. Among the 1,740 and 1,389 original GIs from CRISPR and shRNA screening, 1,699 and 1,368 GIs remained after mapping SPs on the PPI networks. Similar to the KEGG network RPs, the RPs were also applied to the initial GIs of the mutated genes whose number of SPs was two or more in the PPI networks, where the number of initial GIs was 789 and 570 in CRISPR and shRNA screenings. The results are described in FIG. 9 (for clarity, FIG. 9 contains only the results of the mutated genes whose number of initial GIs is five or more; the y-axis is log-scale, and the x-axis indicates each mutated gene; INIT: the initial GIs after mapping on the PPI networks (no RP was applied); RP2: GIs after applying RP2 to the initial GIs. RP1: GIs after applying RP1 to the initial GIs).

As seen in FIG. 9, the 789 initial GIs were narrowed down to 607 and 183 GIs for CRISPR screenings, and the 570 initial GIs were narrowed down to 497 and 142 GIs for shRNA screening with RP2 and RP1, respectively. The present inventors found that the initial GIs were the same as the GIs remaining after RP2 for most mutated genes.

Such finding means that most of the initial SPs were connected within a distance of two on the PPI networks. This is likely because there are some high-degree nodes in the PPI networks (e.g., 994 degrees for TP53 and 1621 degrees for MYC). However, the number of the remaining GIs was greatly decreased by RP1. For example, in the CRISPR screening, 11 out of 18 SPs of the mutated gene PTEN were removed by RP1. Furthermore, for the CCDC57 and HECTD4 mutated genes, there were no remaining SPs when RP1 was applied, which indicates that the SPs do not adjoin each other at all.

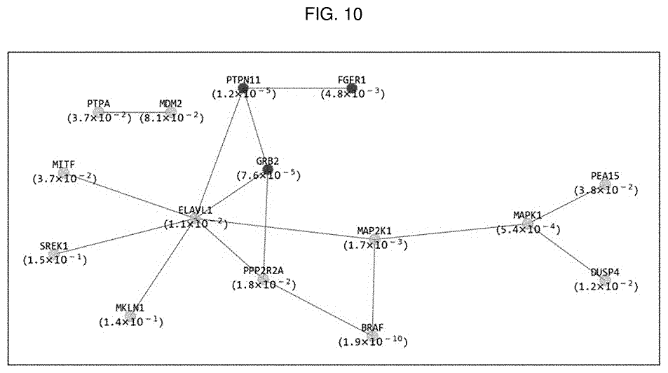

The SPNs for the PPI networks for each mutated gene were also constructed in the same manner as those for the KEGG networks. For example, SPN1 for mutated BRAF from CRISPR screening is depicted in FIG. 10 (The red and blue nodes are the SPs associated with GIs sensitive and resistant to mutated BRAF, respectively; for clarity, SPN1 including only the SPs that adjoin each other is represented while SPN2 is not presented as it was complex).

The SPN1 consists of the 12 sensitive (red nodes) and three resistant (blue nodes) SPs for mutated BRAF. In the present disclosure, it was observed that there was one large single network composed of 13 SPs and one separated edge connecting two SPs, i.e., PTPA and MDM2.

In addition, it was confirmed that ELAVL1 was connected to six other SPs (i.e., it had the highest degree in the network), so it can be considered an important therapeutic target. In fact, ELAVL1 knockdown led to suppression of the proliferation of melanoma cells with mutated BRAF (V600E), which is consistent with the experimental results of the present disclosure that ELAVL1 is a sensitive SP for mutated BRAF. Furthermore, ELAVL1 has been researched as a therapeutic target for various other cancers, such as colorectal, breast, and ovarian cancers.

3-4. Evaluation with SynlethDB and MISL

The results were compared to the two kinds of datasets containing synthetic lethal interactions (SLI). Synthetic lethal interaction (SLI) is a type of GI between two genes such that simultaneous perturbations of the two genes result in cell death, while a perturbation of either gene alone is not lethal.

The first dataset is synlethDB, which is a comprehensive database that contains SLIs collected from biochemical assays, other related databases, computational predictions, and text mining results on human species. All 16,926 SLIs reported in synlethDB were used for the evaluation.

The second dataset is the SLIs characterized by the MiSL algorithm, whose underlying assumption is that the synthetic lethal partner of a mutation will be amplified more frequently or deleted less frequently in cancer cells harboring the mutation, which yielded 119,548 SLIs in total. Given the definition of SLI, the sensitive GIs were only compared to the two kinds of datasets.

First, the sensitive GIs characterized with the exclusion procedure (SGWEs) and without the exclusion procedure (SGOEs) were evaluated with recall and precision measures. The 418 SGWEs and 389 SGOEs from CRISPR screening were evaluated for each of the two datasets, and their results were compared.

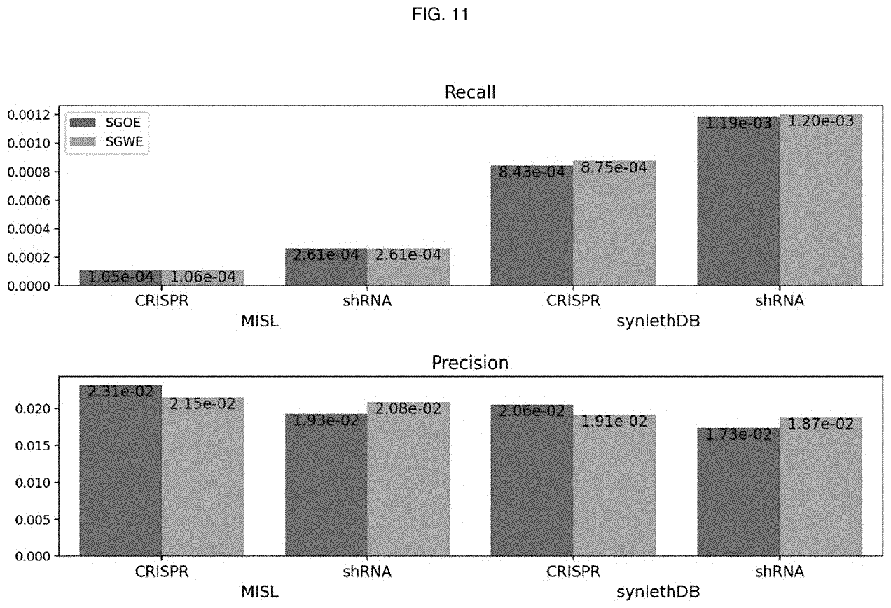

In the same manner, 480 SGWEs and 519 SGOEs from shRNA screening were assessed. As can be seen in FIG. 11, there were no significant performance differences between SGWEs and SGOEs. In terms of recall, it was observed that SGWEs produced slightly better performance for all comparisons, except for shRNA screening evaluated on the MISL datasets. In terms of precision, the SGWEs showed slightly better performance in the shRNA screening and slightly worse performance in the CRISPR screening. Given the evaluation results, the present inventors concluded that the use of the exclusion procedure does not yield any significant difference.

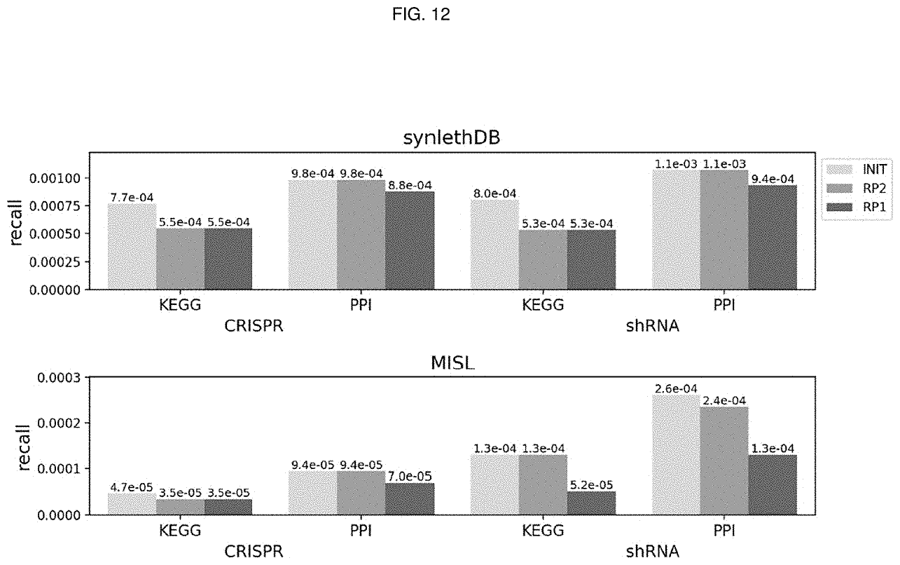

Second, the sensitive GIs determined without application of any of RP (INIT), RP2, and RP1 were evaluated with recall and precision measures, and the results were compared and are depicted in FIG. 12. As shown in FIG. 12, in most comparisons, the recall decreased as the RPs were applied, which was expected because the characterized GIs were narrowed down by applying RP2 and RP1 (i.e., the GIs by RP2 are a subset of those by INIT, and the GIs by RP1 are a subset of those by RP2).

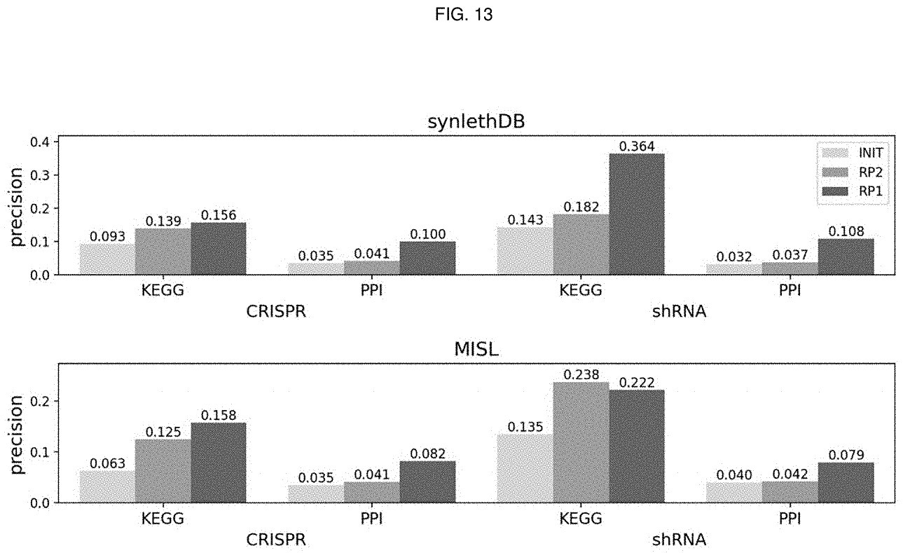

However, as can be seen in FIG. 13, the precision tends to increase in the order of INIT, RP2, and RP1 in most comparisons. FIG. 13 depicts the precision of the sensitive GIs with no RP, RP2, and RP1. The sensitive GIs refined based on KEGG/PPI networks were evaluated with the SLIs in synlethDB and MISL. For most comparisons, RP1 exhibited the highest precision, followed by RP2 and then INIT (in the figure, INIT stands for the initial sensitive GIs after mapping on the molecular networks (no RP was applied); RP2 for the sensitive GIs after applying RP2 to the initial GIs; RP1 for the sensitive GIs after applying RP1 to the initial GIs; RP for refining process; and GI for genetic interaction).

One exception is the evaluation with MISL for the sensitive GIs from shRNA screening refined by KEGG network analysis, where the precision of RP2 was higher than that of RP1. According to the evaluation results, the present inventors concluded that the precision is enhanced by applying the RPs devised in the present disclosure.

It was observed that the GIs mapped on the KEGG and PPI networks (i.e., the initial GIs) had higher precision than those before applying the mapping process (i.e., the original GIs). With the original sensitive GIs, the precision ranged from 0.019 to 0.022 for the four comparisons (see FIG. 11). On the other hand, with the initial sensitive GIs, the precision ranged from 0.032 to 0.093 for the eight comparisons (see FIG. 12). The fact that many cancer-related genes are included in the KEGG and PPI networks could be one of the reasons for the better performance. In addition, it was confirmed that the precision was higher for the sensitive GIs refined on the KEGG networks (0.063˜0.364) than those refined on the PPI networks (0.032˜0.108). This is because the KEGG networks contain manually curated interactions that are considerably researched and well established.

### 4. DISCUSSION AND CONCLUSIONS

As one of the applications of the SPNs, comparing SPNs between KEGG and PPI networks of a certain mutated gene can reveal its therapeutic potential in more detail. For example, SPN1 based on the PPI network for mutated BRAF from CRISPR screening contained 15 interactions among 15 SPs, while SPN1 based on the KEGG network contained 6 interactions among 7 SPs.

The present inventors noticed that the edge between PTPN11 and MAPK1 in the KEGG network was connected with a path (PTPN11-ELAVL1-MAP2K1-MAPK1) in the PPI networks. In other words, the present inventors specified a therapeutic path for mutated BRAF that should be more effective because it is supported by both the KEGG and PPI networks.

In addition, comparing SPNs between KEGG and PPI networks provides some interesting observations. For example, it was observed that PPP2R2A was removed from SPN2 for the KEGG networks, but was contained in SPN1 for the PPI networks, as it directly interacts with other SPs. This was possible because there are much denser interactions in PPI networks than in KEGG networks.

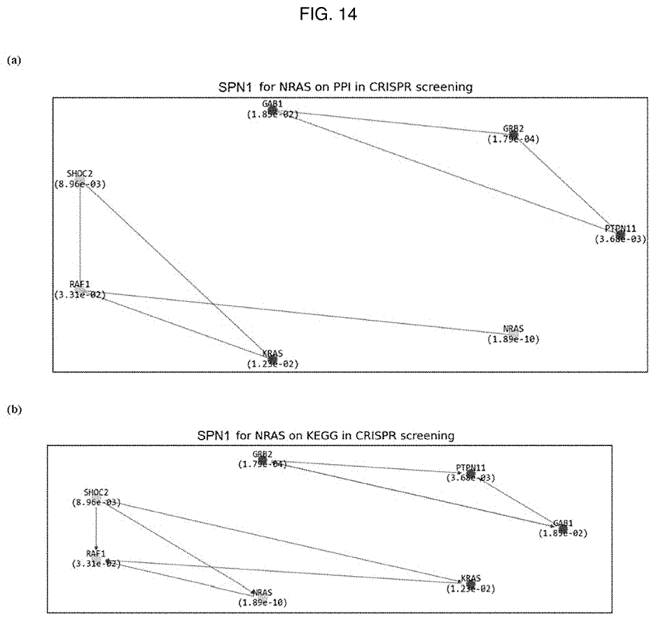

On the other hand, as another example, SPN1 for the PPI network for mutated NRAS contained seven interactions among seven SPs as shown in FIG. 14 (see FIG. 14a). Interestingly, it was identical to SPN1 for the KEGG networks, except for the interaction between SHOC2 and NRAS in the KEGG analysis (see FIG. 14b).

The greater number of edges in the KEGG network was unexpected because PPI networks are more densely connected than KEGG networks. One possible explanation is that there are considerable indirect interactions (i.e., not physical interactions) in KEGG networks.

In several previous studies, genetic interactions were also generated from high-throughput experiments by incorporating biological networks, such as PPI networks and pathway databases. However, their strategies of using biological networks are somewhat different from the present disclosure. For example, in the previous studies, biological networks were mainly applied to a pair of genes, a certain mutated gene and its single SP. On the other hand, in this study, biological networks were applied to all SPs of a certain mutated gene together, except for the mutated gene itself. In other words, the present disclosure newly considers the connectedness of SPs for each mutated gene on biological networks, which was not addressed in previous work.

Therefore, the present inventors speculate that our methods could provide new aspects of GI characterization. In vivo and in vitro experiments, which consume a lot of time and costs, require a handful of therapeutic candidates that are potentially correct. From that perspective, the results of the present disclosure could provide assistance to in vivo and in vitro experiments and are expected to guide research on cancer therapeutics.

What is claimed is:

1. A method for analyzing a genetic interaction, the method comprising:
a first profile input step of inputting a gene dataset including at least one profile selected from the group consisting of a mutation profile, a loss-of-function profile, and an expression profile, for at least one gene cluster associated with at least one type of cells;
a second profile input step of inputting a network set;
a data mapping step of mapping the gene dataset to a network set to generate genetic interaction data; and
a refining step of excluding a synthetic partner for a specific mutant gene from the network profile in the genetic interaction data when the synthetic partner for the specific mutant gene is not located within a predetermined genetic distance from a different synthetic partner for the specific mutant gene on the genetic interaction data.

2. The method of claim 1, wherein the gene dataset of the first profile input step is inputted from a DepMap database.

3. The method of claim 1, wherein the network set comprises at least one selected from the group consisting of a genetic interaction network profile and a protein-protein interaction network profile.

4. The method of claim 3, wherein the genetic interaction network profile is inputted from at least one database selected from the group consisting of KEGG pathway, SIGnature DataBase, Gene ontology, Consortium, DisGeNET, and Diseases.

5. The method of claim 3, wherein the protein-protein interaction (PPI) network profile is inputted from BIOGRID database.

6. The method of claim 1, wherein the mapping comprises a process of excluding a depletion score for a non-expressed gene from the mapping by using the expression profile.

7. The method of claim 1, wherein the predetermined genetic distance may be at least one selected from the group consisting of 1, 2, 3, 4, and 5.

8. The method of claim 1, wherein the method further comprises a target gene deriving step for deriving, as a potential therapeutic target gene, a gene interacting with a certain gene on the genetic interaction data to which the refining step has been applied.

9. A system for analyzing a genetic interaction, the system comprising at least one processor that operates to execute computer-readable instructions, wherein the at least one processor is adapted to:
receive a gene dataset, including at least one profile selected from the group consisting of a mutation profile a loss-of-function profile, and an expression profile, for at least one gene cluster associated with at least one type of cells;
receive a network set;
map the gene dataset to the network set to generate genetic interaction data; and
exclude a synthetic partner for a specific mutant gene from the network profile in the genetic interaction data when the synthetic partner for the specific mutant gene is not located within a predetermined genetic distance from a different synthetic partner for the specific mutant gene on the genetic interaction data.

10. A computer program recorded on a computer-readable medium to implement a method for analyzing a genetic interaction, the method comprising:
a first profile input step of inputting a gene dataset including at least one profile selected from the group consisting of a mutation profile, a loss-of-function profile, and an expression profile, for at least one gene cluster associated with at least one type of cells;
a second profile input step of inputting a network set;
a data mapping step of mapping the gene dataset to a network set to generate genetic interaction data; and
a refining step of excluding a synthetic partner for a specific mutant gene from the network profile in the genetic interaction data when the synthetic partner for the specific mutant gene is not located within a predetermined genetic distance from a different synthetic partner for the specific mutant gene on the genetic interaction data.
